(19) United States
(12) Patent Application Publication
(10) Pub. No.: US 2016/0108432 A1
(43) Pub. Date: Apr. 21, 2016

(54) DROPLET ACTUATOR FOR ELECTROPORATION AND TRANSFORMING CELLS

(71) Applicant: ADVANCED LIQUID LOGIC, INC., San Diego, CA (US)

(72) Inventors: Srikoundinya Punnamaraju, Morrisville, NC (US); Melissa A. Sandahl, Morrisville, NC (US); John J. Harrington, Clayton, NC (US); Allen E. Eckhardt, San Diego, CA (US); Zhishan Hua, Oceanside, CA (US); Michael G. Pollack, Durham, CA (US)

(73) Assignee: Advanced Liquid Logic, Inc., San Diego, CA (US)

(21) Appl. No.: 14/890,451

(22) PCT Filed: May 16, 2014
(86) PCT No.: PCT/US14/38374

§ 371 Date: Nov. 11, 2015

Related U.S. Application Data
(60) Provisional application No. 61/824,183, filed on May 16, 2013.

(51) Int. Cl. C12N 15/87; C12M 1/42; C12N 13/00

(52) U.S. Cl. CPC C12N 15/87; C12N 13/00; C12M 35/02

(57) ABSTRACT

The invention provides a droplet actuator designed for performing electroporation on cells in droplets. The invention also provides method and systems for performing electroporation on cells in droplets on a droplet actuator.

### RELATED APPLICATIONS

In addition to the patent applications cited herein, each of which is incorporated herein by reference, this patent application is related to and claims priority to U.S. Provisional Patent Application No. 61/824,183, filed on May 16, 2013, entitled “Droplet Actuator for Electroporation and Transforming Cells;” the entire disclosure of which is incorporated herein by reference.

### GOVERNMENT INTEREST

This invention was made with government support under HR0011-12-C-0057 awarded by Defense Advanced Research Projects Agency. The United States Government has certain rights in the invention.

### FIELD OF THE INVENTION

The present invention generally relates to droplet actuators and methods for their use. In particular, the present invention provides a droplet actuator designed for performing electroporation on cells in droplets.

### BACKGROUND

Droplet actuators are used to conduct a wide variety of droplet operations, such as droplet transport and droplet dispensing. A droplet actuator typically includes one or more substrates configured to form a surface or gap for conducting droplet operations. The one or more substrates establish a droplet operations surface or gap for conducting droplet operations and may also include electrodes arranged to conduct the droplet operations. The droplet operations substrate or the gap between the substrates may be coated or filled with a filler fluid that is immiscible with the liquid that forms the droplets.

In one application, samples within droplet actuators may include cells to be manipulated, such as for the incubation and growth of cells within droplet actuators. It may be advantageous to introduce substances into such cells within a droplet actuator, such as molecular probes, chemical agents, proteins, or nucleic acids. Therefore, there is a need for droplet actuator designs and techniques for performing electroporation on cells in droplets.

### BRIEF DESCRIPTION

A droplet actuator is provided, comprising: a) at least one substrate, wherein droplet operations electrodes are associated with the at least one substrate; and b) one or more electroporation electrodes. In one embodiment the droplet actuator is open at the top. In another embodiment, a droplet operations gap is formed from a single substrate folded on itself. In a further embodiment, a droplet operations gap is formed from a top substrate and a bottom substrate separated by the droplet operations gap. In another embodiment, the one or more electroporation electrodes are substantially flat. In yet another embodiment, the one or more electroporation electrodes comprise a conductive material, particularly wherein the conductive material is a metal, and more particularly wherein the metal is copper or gold.

In certain embodiments, the droplet actuator comprises one or more electroporation electrodes situated atop one or more droplet operations electrodes. In one embodiment, the one or more electroporation electrodes are coupled to a power source via one or more conductive paths, particularly wherein the power source is an electroporation pulse generator. In another embodiment, the one or more electroporation electrodes comprise radially oriented arms. In a further embodiment, the one or more electroporation electrodes comprise a serpentine shape. In yet another embodiment, the one or more electroporation electrodes overlap one or more droplet operations electrodes, particularly wherein the one or more electroporation electrodes overlap a droplet operations electrode and portions of adjacent droplet operations electrodes. In another embodiment, the one or more electroporation electrodes are arranged to allow the droplet operations electrodes to perform electrowetting mediated droplet operations on one or more droplets.

In another embodiment, a droplet actuator comprising: a) a top substrate and a bottom substrate separated to form a droplet operations gap; b) droplet operations electrodes atop the bottom substrate facing the droplet operations gap; c) a via extending into the bottom substrate, whereby the droplet operations electrodes are electrically coupled to a power source; d) a dielectric layer atop the droplet operations electrodes and atop the bottom substrate in areas between the droplet operations electrodes; e) an electroporation electrode atop the dielectric layer, wherein the electroporation electrode comprises a footprint; and f) a hydrophobic coating atop the dielectric material surrounding the footprint of the electroporation electrode. In one embodiment, the bottom substrate and/or the top substrate are formed of a dielectric material, particularly wherein the dielectric materials is selected from the group consisting of PCB, plastic, glass, and a semiconductor material. In another embodiment, the droplet actuator further comprises a conductive layer atop the top substrate, wherein the conductive layer faces the droplet operations gap. In a further embodiment, the droplet actuator further comprises a hydrophobic layer atop the conductive layer.

A method of producing electroporation is also provided, comprising: a) situating a droplet comprising cells atop an electroporation electrode in a droplet actuator, wherein the electroporation electrode is covered with a hydrophobic coating; and b) delivering a pulse to the electroporation electrode, thereby causing electroporation of the cells in the droplet. In some embodiments, the droplet is surrounded by oil, substantially surrounded by oil, or is floating in oil. In other embodiments, the method further comprises transporting the droplet onto and/or away from the electroporation electrode using electrowetting mediated droplet operations along droplet operations electrodes. In a further embodiment, the droplet is pinned on the electroporation electrode and transporting the droplet away from the electroporation electrode comprises adjusting activation of the droplet operations electrodes to a frequency sufficient to cause oscillations in the droplet that reverse pinning, particularly wherein the frequency is about 2 Hz. In another embodiment, the droplet is pinned on the electroporation electrode and transporting the droplet away from the electroporation electrode comprises adjusting the transport rate to a rate sufficient to reverse pinning, particularly wherein the transport rate is reduced to greater than about 1 second, about 5 seconds, about 10 seconds, about 20 seconds, or about 30 seconds. In yet another embodiment, transport failures to and/or from the electroporation electrode are mitigated by using a droplet about two times the size of the footprint of the electrowetting electrode, particularly wherein the size of the droplet is about 700 nL. In a further embodiment, electrowetting is used to retain the droplet in place during delivery of the pulse to the electroporation electrode to prevent the droplet from floating away.

In some embodiments, following electroporation of the cells in the droplet, the droplet is transported away from the electroporation electrode for downstream processing using electrowetting mediated droplet operations. In one embodiment, downstream processing comprises merging the droplet with a droplet comprising recovery media, thereby producing a combined droplet. In another embodiment, the combined droplet is removed from the droplet actuator via a recovery port, particularly wherein the recovery port comprises an opening in a top substrate or a bottom substrate of the droplet actuator, or an opening in a sidewall of the droplet actuator. In a further embodiment, downstream processing comprises splitting the droplet into two or more daughter droplets; determining that each daughter droplet comprises a single cell; and merging each daughter droplet with a droplet comprising culture medium, thereby producing combined droplets. In yet another embodiment, each combined droplet is incubated, thereby producing an incubated droplet. In other embodiments, the incubated droplet is split into two or more daughter droplets for sampling, thereby producing one or more sample droplets, particularly wherein the one or more sample droplets are assayed and/or tested, and more particularly wherein the one or more sample droplets are tested to determine whether cells were successfully transformed by electroporation. In another embodiment, the incubated droplet is refreshed with culture media by merging the incubated droplet with a droplet comprising culture media using electrowetting mediated droplet operations.

A method for multiplex automated genome engineering (MAGE) is also provided, comprising repeated introduction of synthetic DNA into cells in a droplet, wherein the synthetic DNA is introduced into the cells using any of the methods of producing electroporation disclosed herein.

### BRIEF DESCRIPTION OF THE DRAWINGS

FIG. 1 illustrates a portion of a droplet operations electrode layout of a droplet actuator for performing electroporation on cells in droplets;

FIG. 2 illustrates a variation of a portion of a droplet operations electrode layout of a droplet actuator as shown in FIG. 1 in which the electroporation electrode has radially oriented arms;

FIG. 3 illustrates a variation of a portion of a droplet operations electrode layout of a droplet actuator as shown in FIG. 1 in which the electroporation electrode has a serpentine shape; and

FIG. 4 illustrates another variation of a portion of a droplet operations electrode layout of a droplet actuator as shown in FIG. 1 in which the electroporation electrode has a serpentine shape.

### DEFINITIONS

As used herein, the following terms have the meanings indicated.

“Activate,” with reference to one or more droplet operations electrodes, means affecting a change in the electrical state of the one or more electrodes which, in the presence of a droplet, results in a droplet operation. Activation of an electrode can be accomplished using alternating or direct current. Any suitable voltage may be used. For example, an electrode may be activated using a voltage which is greater than about 150 V, or greater than about 200 V, or greater than about 250 V, or from about 275 V to about 1000 V, or about 300 V. Where alternating current is used, any suitable frequency may be employed. For example, an electrode may be activated using alternating current having a frequency from about 1 Hz to about 10 MHz, or from about 10 Hz to about 60 Hz, or from about 20 Hz to about 40 Hz, or about 30 Hz. “Activate,” with reference to an electroporation electrode means to apply an electroporation pulse to the electrode.

“Bead,” with respect to beads on a droplet actuator, means any bead or particle that is capable of interacting with a droplet on or in proximity with a droplet actuator. Beads may be any of a wide variety of shapes, such as spherical, generally spherical, egg shaped, disc shaped, cubical, amorphous and other three dimensional shapes. The bead may, for example, be capable of being subjected to a droplet operation in a droplet on a droplet actuator or otherwise configured with respect to a droplet actuator in a manner which permits a droplet on the droplet actuator to be brought into contact with the bead on the droplet actuator and/or off the droplet actuator. Beads may be provided in a droplet, in a droplet operations gap, or on a droplet operations surface. Beads may be provided in a reservoir that is external to a droplet operations gap or situated apart from a droplet operations surface, and the reservoir may be associated with a flow path that permits a droplet including the beads to be brought into a droplet operations gap or into contact with a droplet operations surface. Beads may be manufactured using a wide variety of materials, including for example, resins, and polymers. The beads may be any suitable size, including for example, microbeads, microparticles, nanobeads and nanoparticles. In some cases, beads are magnetically responsive; in other cases beads are not significantly magnetically responsive. For magnetically responsive beads, the magnetically responsive material may constitute substantially all of a bead, a portion of a bead, or only one component of a bead. The remainder of the bead may include, among other things, polymeric material, coatings, and moieties which permit attachment of an assay reagent. Examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® particles, available from Invitrogen Group, Carlsbad, Calif.), fluorescent microparticles and nanoparticles, coated magnetic microparticles and nanoparticles, ferromagnetic microparticles and nanoparticles, coated ferromagnetic microparticles and nanoparticles, and those described in U.S. Patent Publication Nos. 20050260686, entitled “Multiplex flow assays preferably with magnetic particles as solid phase,” published on Nov. 24, 2005; 20030132538, entitled “Encapsulation of discrete quanta of fluorescent particles,” published on Jul. 17, 2003; 20050118574, entitled “Multiplexed Analysis of Clinical Specimens Apparatus and Method,” published on Jun. 2, 2005; 20050277197. Entitled “Microparticles with Multiple Fluorescent Signals and Methods of Using Same,” published on Dec. 15, 2005; 20060159962, entitled “Magnetic Microspheres for use in Fluorescence-based Applications,” published on Jul. 20, 2006; the entire disclosures of which are incorporated herein by reference for their teaching concerning beads and magnetically responsive materials and beads. Beads may be pre-coupled with a biomolecule or other substance that is able to bind to and form a complex with a biomolecule. Beads may be pre-coupled with an antibody, protein or antigen, DNA/RNA probe or any other molecule with an affinity for a desired target. Examples of droplet actuator techniques for immobilizing magnetically responsive beads and/or non-magnetically responsive beads and/or conducting droplet operations protocols using beads are described in U.S. patent application Ser. No. 11/639,566, entitled “Droplet-Based Particle Sorting,” filed on Dec. 15, 2006; U.S. Patent Application No. 61/039,183, entitled “Multiplexing Bead Detection in a Single Droplet,” filed on Mar. 25, 2008; U.S. Patent Application No. 61/047,789, entitled “Droplet Actuator Devices and Droplet Operations Using Beads,” filed on Apr. 25, 2008; U.S. Patent Application No. 61/086,183, entitled “Droplet Actuator Devices and Methods for Manipulating Beads,” filed on Aug. 5, 2008; International Patent Application No. PCT/US2008/053545, entitled “Droplet Actuator Devices and Methods Employing Magnetic Beads,” filed on Feb. 11, 2008; International Patent Application No. PCT/US2008/058018, entitled “Bead-based Multiplexed Analytical Methods and Instrumentation,” filed on Mar. 24, 2008; International Patent Application No. PCT/US2008/058047, “Bead Sorting on a Droplet Actuator,” filed on Mar. 23, 2008; and International Patent Application No. PCT/US2006/047486, entitled “Droplet-based Biochemistry,” filed on Dec. 11, 2006; the entire disclosures of which are incorporated herein by reference. Bead characteristics may be employed in the multiplexing aspects of the invention. Examples of beads having characteristics suitable for multiplexing, as well as methods of detecting and analyzing signals emitted from such beads, may be found in U.S. Patent Publication No. 20080305481, entitled “Systems and Methods for Multiplex Analysis of PCR in Real Time,” published on Dec. 11, 2008; U.S. Patent Publication No. 20080151240, “Methods and Systems for Dynamic Range Expansion,” published on Jun. 26, 2008; U.S. Patent Publication No. 20070207513, entitled “Methods, Products, and Kits for Identifying an Analyte in a Sample,” published on Sep. 6, 2007; U.S. Patent Publication No. 20070064990, entitled “Methods and Systems for Image Data Processing,” published on Mar. 22, 2007; U.S. Patent Publication No. 20060159962, entitled “Magnetic Microspheres for use in Fluorescence-based Applications,” published on Jul. 20, 2006; U.S. Patent Publication No. 20050277197, entitled “Microparticles with Multiple Fluorescent Signals and Methods of Using Same,” published on Dec. 15, 2005; and U.S. Patent Publication No. 20050118574, entitled “Multiplexed Analysis of Clinical Specimens Apparatus and Method,” published on Jun. 2, 2005. Droplets subjected to an electroporation pulse pursuant to the invention may include beads.

“Droplet” means a volume of liquid on a droplet actuator. Typically, a droplet is at least partially bounded by a filler fluid. For example, a droplet may be completely surrounded by a filler fluid or may be bounded by filler fluid and one or more surfaces of the droplet actuator. As another example, a droplet may be bounded by filler fluid, one or more surfaces of the droplet actuator, and/or the atmosphere. As yet another example, a droplet may be bounded by filler fluid and the atmosphere. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, combinations of such shapes, and various shapes formed during droplet operations, such as merging or splitting or formed as a result of contact of such shapes with one or more surfaces of a droplet actuator. For examples of droplet fluids that may be subjected to droplet operations using the approach of the invention, see International Patent Application No. PCT/US 06/47486, entitled, “Droplet-Based Biochemistry,” filed on Dec. 11, 2006. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. Moreover, a droplet may include a reagent, such as water, deionized water, saline solutions, acidic solutions, basic solutions, detergent solutions and/or buffers. Other examples of droplet contents include reagents, such as a reagent for a biochemical protocol, such as a nucleic acid amplification protocol, an affinity-based assay protocol, an enzymatic assay protocol, a sequencing protocol, and/or a protocol for analyses of biological fluids. A droplet may include one or more beads. A droplet may me subjected to an electroporation pulse.

“Droplet Actuator” means a device for manipulating droplets. For examples of droplet actuators, see Pamula et al., U.S. Pat. No. 6,911,132, entitled “Apparatus for Manipulating Droplets by Electrowetting-Based Techniques,” issued on Jun. 28, 2005; Pamula et al., U.S. patent application Ser. No. 11/343,284, entitled “Apparatuses and Methods for Manipulating Droplets on a Printed Circuit Board,” filed on filed on Jan. 30, 2006; Pollack et al., International Patent Application No. PCT/US2006/047486, entitled “Droplet-Based Biochemistry,” filed on Dec. 11, 2006; Shenderov, U.S. Pat. No. 6,773,566, entitled “Electrostatic Actuators for Microfluidics and Methods for Using Same,” issued on Aug. 10, 2004 and U.S. Pat. No. 6,565,727, entitled “Actuators for Microfluidics Without Moving Parts,” issued on Jan. 24, 2000; Kim and/or Shah et al., U.S. patent application Ser. No. 10/343,261, entitled “Electrowetting-driven Micropumping,” filed on Jan. 27, 2003, Ser. No. 11/275,668, entitled “Method and Apparatus for Promoting the Complete Transfer of Liquid Drops from a Nozzle,” filed on Jan. 23, 2006, Ser. No. 11/460,188, entitled “Small Object Moving on Printed Circuit Board,” filed on Jan. 23, 2006, Ser. No. 12/465,935, entitled “Method for Using Magnetic Particles in Droplet Microfluidics,” filed on May 14, 2009, and Ser. No. 12/513,157, entitled “Method and Apparatus for Real-time Feedback Control of Electrical Manipulation of Droplets on Chip,” filed on Apr. 30, 2009; Velev, U.S. Pat. No. 7,547,380, entitled “Droplet Transportation Devices and Methods Having a Fluid Surface,” issued on Jun. 16, 2009; Sterling et al., U.S. Pat. No. 7,163,612, entitled “Method, Apparatus and Article for Microfluidic Control via Electrowetting, for Chemical, Biochemical and Biological Assays and the Like,” issued on Jan. 16, 2007; Becker and Gascoyne et al., U.S. Pat. No. 7,641,779, entitled “Method and Apparatus for Programmable fluidic Processing,” issued on Jan. 5, 2010, and U.S. Pat. No. 6,977,033, entitled “Method and Apparatus for Programmable fluidic Processing,” issued on Dec. 20, 2005; Decre et al., U.S. Pat. No. 7,328,979, entitled “System for Manipulation of a Body of Fluid,” issued on Feb. 12, 2008; Yamakawa et al., U.S. Patent Pub. No. 20060039823, entitled “Chemical Analysis Apparatus,” published on Feb. 23, 2006; Wu, International Patent Pub. No. WO/2009/003184, entitled “Digital Microfluidics Based Apparatus for Heat-exchanging Chemical Processes,” published on Dec. 31, 2008; Fouillet et al., U.S. Patent Pub. No. 20090192044, entitled “Electrode Addressing Method,” published on Jul. 30, 2009; Fouillet et al., U.S. Pat. No. 7,052,244, entitled “Device for Displacement of Small Liquid Volumes Along a Micro-catenary Line by Electrostatic Forces,” issued on May 30, 2006; Marchand et al., U.S. Patent Pub. No. 20080124252, entitled “Droplet Microreactor,” published on May 29, 2008; Adachi et al., U.S. Patent Pub. No. 20090321262, entitled “Liquid Transfer Device,” published on Dec. 31, 2009; Roux et al., U.S. Patent Pub. No. 20050179746, entitled “Device for Controlling the Displacement of a Drop Between two or Several Solid Substrates,” published on Aug. 18, 2005; Dhindsa et al., “Virtual Electrowetting Channels: Electronic Liquid Transport with Continuous Channel Functionality,” Lab Chip, 10:832-836 (2010); the entire disclosures of which are incorporated herein by reference, along with their priority documents. Certain droplet actuators will include one or more substrates arranged with a droplet operations gap therebetween and electrodes associated with (e.g., layered on, attached to, and/or embedded in) the one or more substrates and arranged to conduct one or more droplet operations. For example, certain droplet actuators will include a base (or bottom) substrate, droplet operations electrodes associated with the substrate, one or more dielectric layers atop the substrate and/or electrodes, and optionally one or more hydrophobic layers atop the substrate, dielectric layers and/or the electrodes forming a droplet operations surface. A top substrate may also be provided, which is separated from the droplet operations surface by a gap, commonly referred to as a droplet operations gap. Various electrode arrangements on the top and/or bottom substrates are discussed in the above-referenced patents and applications and certain novel electrode arrangements are discussed in the description of the invention. During droplet operations it is preferred that droplets remain in continuous contact or frequent contact with a ground or reference electrode. A ground or reference electrode may be associated with the top substrate facing the gap, the bottom substrate facing the gap, in the gap. Where electrodes are provided on both substrates, electrical contacts for coupling the electrodes to a droplet actuator instrument for controlling or monitoring the electrodes may be associated with one or both plates. In some cases, electrodes on one substrate are electrically coupled to the other substrate so that only one substrate is in contact with the droplet actuator. In one embodiment, a conductive material (e.g., an epoxy, such as MASTER BOND™ Polymer System EP79, available from Master Bond, Inc., Hackensack, N.J.) provides the electrical connection between electrodes on one substrate and electrical paths on the other substrates, e.g., a ground electrode on a top substrate may be coupled to an electrical path on a bottom substrate by such a conductive material. Where multiple substrates are used, a spacer may be provided between the substrates to determine the height of the gap therebetween and define dispensing reservoirs. The spacer height may, for example, be from about 5 μm to about 600 μm, or about 100 μm to about 400 μm, or about 200 μm to about 350 μm, or about 250 μm to about 300 μm, or about 275 μm. The spacer may, for example, be formed of a layer of projections form the top or bottom substrates, and/or a material inserted between the top and bottom substrates. One or more openings may be provided in the one or more substrates for forming a fluid path through which liquid may be delivered into the droplet operations gap. The one or more openings may in some cases be aligned for interaction with one or more electrodes, e.g., aligned such that liquid flowed through the opening will come into sufficient proximity with one or more droplet operations electrodes to permit a droplet operation to be effected by the droplet operations electrodes using the liquid. The base (or bottom) and top substrates may in some cases be formed as one integral component. One or more reference electrodes may be provided on the base (or bottom) and/or top substrates and/or in the gap. Examples of reference electrode arrangements are provided in the above referenced patents and patent applications. In various embodiments, the manipulation of droplets by a droplet actuator may be electrode mediated, e.g., electrowetting mediated or dielectrophoresis mediated or Coulombic force mediated. Examples of other techniques for controlling droplet operations that may be used in the droplet actuators of the invention include using devices that induce hydrodynamic fluidic pressure, such as those that operate on the basis of mechanical principles (e.g. external syringe pumps, pneumatic membrane pumps, vibrating membrane pumps, vacuum devices, centrifugal forces, piezoelectric/ultrasonic pumps and acoustic forces); electrical or magnetic principles (e.g. electroosmotic flow, electrokinetic pumps, ferrofluidic plugs, electrohydrodynamic pumps, attraction or repulsion using magnetic forces and magnetohydrodynamic pumps); thermodynamic principles (e.g. gas bubble generation/phase-change-induced volume expansion); other kinds of surface-wetting principles (e.g. electrowetting, and optoelectrowetting, as well as chemically, thermally, structurally and radioactively induced surface-tension gradients); gravity; surface tension (e.g., capillary action); electrostatic forces (e.g., electroosmotic flow); centrifugal flow (substrate disposed on a compact disc and rotated); magnetic forces (e.g., oscillating ions causes flow); magnetohydrodynamic forces; and vacuum or pressure differential. In certain embodiments, combinations of two or more of the foregoing techniques may be employed to conduct a droplet operation in a droplet actuator of the invention. Similarly, one or more of the foregoing may be used to deliver liquid into a droplet operations gap, e.g., from a reservoir in another device or from an external reservoir of the droplet actuator (e.g., a reservoir associated with a droplet actuator substrate and a flow path from the reservoir into the droplet operations gap). Droplet operations surfaces of certain droplet actuators of the invention may be made from hydrophobic materials or may be coated or treated to make them hydrophobic. For example, in some cases some portion or all of the droplet operations surfaces may be derivatized with low surface-energy materials or chemistries, e.g., by deposition or using in situ synthesis using compounds such as poly- or per-fluorinated compounds in solution or polymerizable monomers. Examples include TEFLON® AF (available from DuPont, Wilmington, Del.), members of the cytop family of materials, coatings in the FLUOROPEL® family of hydrophobic and superhydrophobic coatings (available from Cytonix Corporation, Beltsville, Md.), silane coatings, fluorosilane coatings, hydrophobic phosphonate derivatives (e.g., those sold by Aculon, Inc), and NOVEC™ electronic coatings (available from 3M Company, St. Paul, Minn.), other fluorinated monomers for plasma-enhanced chemical vapor deposition (PECVD), and organosiloxane (e.g., SiOC) for PECVD. In some cases, the droplet operations surface may include a hydrophobic coating having a thickness ranging from about 10 nm to about 1,000 nm. Moreover, in some embodiments, the top substrate of the droplet actuator includes an electrically conducting organic polymer, which is then coated with a hydrophobic coating or otherwise treated to make the droplet operations surface hydrophobic. For example, the electrically conducting organic polymer that is deposited onto a plastic substrate may be poly(3,4-ethylenedioxythiophene)poly(styrenesulfonate) (PEDOT:PSS). Other examples of electrically conducting organic polymers and alternative conductive layers are described in Pollack et al., International Patent Application No. PCT/US2010/040705, entitled “Droplet Actuator Devices and Methods,” the entire disclosure of which is incorporated herein by reference. One or both substrates may be fabricated using a printed circuit board (PCB), glass, indium tin oxide (ITO)-coated glass, and/or semiconductor materials as the substrate. When the substrate is ITO-coated glass, the ITO coating is preferably a thickness in the range of about 20 to about 200 nm, preferably about 50 to about 150 nm, or about 75 to about 125 nm, or about 100 nm. In some cases, the top and/or bottom substrate includes a PCB substrate that is coated with a dielectric, such as a polyimide dielectric, which may in some cases also be coated or otherwise treated to make the droplet operations surface hydrophobic. When the substrate includes a PCB, the following materials are examples of suitable materials: MITSUI™ BN-300 (available from MITSUI Chemicals America, Inc., San Jose Calif.); ARLON™ 11N (available from Arlon, Inc, Santa Ana, Calif.).; NELCO® N4000-6 and N5000-30/32 (available from Park Electrochemical Corp., Melville, N.Y.); ISOLA™ FR406 (available from Isola Group, Chandler, Ariz.), especially IS620; fluoropolymer family (suitable for fluorescence detection since it has low background fluorescence); polyimide family; polyester; polyethylene naphthalate; polycarbonate; polyetheretherketone; liquid crystal polymer; cyclo-olefin copolymer (COC); cyclo-olefin polymer (COP); aramid; THERMOUNT® nonwoven aramid reinforcement (available from DuPont, Wilmington, Del.); NOMEX® brand fiber (available from DuPont, Wilmington, Del.); and paper. Various materials are also suitable for use as the dielectric component of the substrate. Examples include: vapor deposited dielectric, such as PARYLENE™ C (especially on glass), PARYLENE™ N, and PARYLENE™ HT (for high temperature, ˜300° C.) (available from Parylene Coating Services, Inc., Katy, Tex.); TEFLON® AF coatings; cytop; soldermasks, such as liquid photoimageable soldermasks (e.g., on PCB) like TAIYO™ PSR4000 series, TAIYO™ PSR and AUS series (available from Taiyo America, Inc. Carson City, Nev.) (good thermal characteristics for applications involving thermal control), and PROBIMER™ 8165 (good thermal characteristics for applications involving thermal control (available from Huntsman Advanced Materials Americas Inc., Los Angeles, Calif.); dry film soldermask, such as those in the VACREL® dry film soldermask line (available from DuPont, Wilmington, Del.); film dielectrics, such as polyimide film (e.g., KAPTON® polyimide film, available from DuPont, Wilmington, Del.), polyethylene, and fluoropolymers (e.g., FEP), polytetrafluoroethylene; polyester; polyethylene naphthalate; cyclo-olefin copolymer (COC); cyclo-olefin polymer (COP); any other PCB substrate material listed above; black matrix resin; polypropylene; and black flexible circuit materials, such as DuPont™ Pyralux® HXC and DuPont™ Kapton® MBC (available from DuPont, Wilmington, Del.). Droplet transport voltage and frequency may be selected for performance with reagents used in specific assay protocols. Design parameters may be varied, e.g., number and placement of on-actuator reservoirs, number of independent electrode connections, size (volume) of different reservoirs, placement of magnets/bead washing zones, electrode size, inter-electrode pitch, and gap height (between top and bottom substrates) may be varied for use with specific reagents, protocols, droplet volumes, etc. In some cases, a substrate of the invention may derivatized with low surface-energy materials or chemistries, e.g., using deposition or in situ synthesis using poly- or per-fluorinated compounds in solution or polymerizable monomers. Examples include TEFLON® AF coatings and FLUOROPEL® coatings for dip or spray coating, other fluorinated monomers for plasma-enhanced chemical vapor deposition (PECVD), and organosiloxane (e.g., SiOC) for PECVD. Additionally, in some cases, some portion or all of the droplet operations surface may be coated with a substance for reducing background noise, such as background fluorescence from a PCB substrate. For example, the noise-reducing coating may include a black matrix resin, such as the black matrix resins available from Toray industries, Inc., Japan. Electrodes of a droplet actuator are typically controlled by a controller or a processor, which is itself provided as part of a system, which may include processing functions as well as data and software storage and input and output capabilities. Reagents may be provided on the droplet actuator in the droplet operations gap or in a reservoir fluidly coupled to the droplet operations gap. The reagents may be in liquid form, e.g., droplets, or they may be provided in a reconstitutable form in the droplet operations gap or in a reservoir fluidly coupled to the droplet operations gap. Reconstitutable reagents may typically be combined with liquids for reconstitution. An example of reconstitutable reagents suitable for use with the invention includes those described in Meathrel, et al., U.S. Pat. No. 7,727,466, entitled “Disintegratable films for diagnostic devices,” granted on Jun. 1, 2010.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles. For examples of droplet operations, see the patents and patent applications cited above under the definition of “droplet actuator.” Impedance or capacitance sensing or imaging techniques may sometimes be used to determine or confirm the outcome of a droplet operation. Examples of such techniques are described in Sturmer et al., U.S. Patent Application Publication No. US20100194408, entitled “Capacitance Detection in a Droplet Actuator,” published on Aug. 5, 2010, the entire disclosure of which is incorporated herein by reference. Generally speaking, the sensing or imaging techniques may be used to confirm the presence or absence of a droplet at a specific electrode. For example, the presence of a dispensed droplet at the destination electrode following a droplet dispensing operation confirms that the droplet dispensing operation was effective. Similarly, the presence of a droplet at a detection spot at an appropriate step in an assay protocol may confirm that a previous set of droplet operations has successfully produced a droplet for detection. Droplet transport time can be quite fast. For example, in various embodiments, transport of a droplet from one electrode to the next may exceed about 1 sec, or about 0.1 sec, or about 0.01 sec, or about 0.001 sec. In one embodiment, the electrode is operated in AC mode but is switched to DC mode for imaging. It is helpful for conducting droplet operations for the footprint area of droplet to be similar to electrowetting area; in other words, 1×-, 2×-3×-droplets are usefully controlled operated using 1, 2, and 3 electrodes, respectively. If the droplet footprint is greater than the number of electrodes available for conducting a droplet operation at a given time, the difference between the droplet size and the number of electrodes should typically not be greater than 1; in other words, a 2× droplet is usefully controlled using 1 electrode and a 3× droplet is usefully controlled using 2 electrodes. When droplets include beads, it is useful for droplet size to be equal to the number of electrodes controlling the droplet, e.g., transporting the droplet.

“Filler fluid” means a fluid associated with a droplet operations substrate of a droplet actuator, which fluid is sufficiently immiscible with a droplet phase to render the droplet phase subject to electrode-mediated droplet operations. For example, the droplet operations gap of a droplet actuator is typically filled with a filler fluid. The filler fluid may, for example, be or include a low-viscosity oil, such as silicone oil or hexadecane filler fluid. The filler fluid may be or include a halogenated oil, such as a fluorinated or perfluorinated oil. The filler fluid may fill the entire gap of the droplet actuator or may coat one or more surfaces of the droplet actuator. Filler fluids may be conductive or non-conductive. Filler fluids may be selected to improve droplet operations and/or reduce loss of reagent or target substances from droplets, improve formation of microdroplets, reduce cross contamination between droplets, reduce contamination of droplet actuator surfaces, reduce degradation of droplet actuator materials, etc. For example, filler fluids may be selected for compatibility with droplet actuator materials. As an example, fluorinated filler fluids may be usefully employed with fluorinated surface coatings. Fluorinated filler fluids are useful to reduce loss of lipophilic compounds, such as umbelliferone substrates like 6-hexadecanoylamido-4-methylumbelliferone substrates (e.g., for use in Krabbe, Niemann-Pick, or other assays); other umbelliferone substrates are described in U.S. Patent Pub. No. 20110118132, published on May 19, 2011, the entire disclosure of which is incorporated herein by reference. Examples of suitable fluorinated oils include those in the Galden line, such as Galden HT170 (bp=170° C., viscosity=1.8 cSt, density=1.77), Galden HT200 (bp=200 C, viscosity=2.4 cSt, d=1.79), Galden HT230 (bp=230 C, viscosity=4.4 cSt, d=1.82) (all from Solvay Solexis); those in the Novec line, such as Novec 7500 (bp=128 C, viscosity=0.8 cSt, d=1.61), Fluorinert FC-40 (bp=155° C., viscosity=1.8 cSt, d=1.85), Fluorinert FC-43 (bp=174° C., viscosity=2.5 cSt, d=1.86) (both from 3M). In general, selection of perfluorinated filler fluids is based on kinematic viscosity (<7 cSt is preferred, but not required), and on boiling point (>150° C. is preferred, but not required, for use in DNA/RNA-based applications (PCR, etc.)). Filler fluids may, for example, be doped with surfactants or other additives. For example, additives may be selected to improve droplet operations and/or reduce loss of reagent or target substances from droplets, formation of microdroplets, cross contamination between droplets, contamination of droplet actuator surfaces, degradation of droplet actuator materials, etc. Composition of the filler fluid, including surfactant doping, may be selected for performance with reagents used in the specific assay protocols and effective interaction or non-interaction with droplet actuator materials. Examples of filler fluids and filler fluid formulations suitable for use with the invention are provided in Srinivasan et al, International Patent Pub. Nos. WO/2010/027894, entitled “Droplet Actuators, Modified Fluids and Methods,” published on Mar. 11, 2010, and WO/2009/021173, entitled “Use of Additives for Enhancing Droplet Operations,” published on Feb. 12, 2009; Sista et al., International Patent Pub. No. WO/2008/098236, entitled “Droplet Actuator Devices and Methods Employing Magnetic Beads,” published on Aug. 14, 2008; and Monroe et al., U.S. Patent Publication No. 20080283414, entitled “Electrowetting Devices,” filed on May 17, 2007; the entire disclosures of which are incorporated herein by reference, as well as the other patents and patent applications cited herein. Fluorinated oils may in some cases be doped with fluorinated surfactants, e.g., Zonyl FSO-100 (Sigma-Aldrich) and/or others. Droplets subjected to an electroporation pulse may be surrounded, substantially surrounded, partially surrounded, and/or floating in a filler fluid.

“Transform,” “transformed” and the like are used broadly to refer to delivery of substances into a cell. The substances are typically nucleic acids, but may also or alternatively include proteins, peptides, and/or other molecules. The term “transform” as used herein broadly includes, without limitation, transformation and transfection, as those terms are used in the art. Electroporation according to any of the embodiments of the invention may result in transformation of cells.

The terms “top,” “bottom,” “over,” “under,” and “on” are used throughout the description with reference to the relative positions of components of the droplet actuator, such as relative positions of top and bottom substrates of the droplet actuator. It will be appreciated that the droplet actuator is functional regardless of its orientation in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface. In one example, filler fluid can be considered as a film between such liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

### DESCRIPTION

The invention provides a droplet actuator designed for performing electroporation on cells in droplets.

### 8.1 DROPLET ACTUATOR WITH ELECTROPORATION ELECTRODE

The droplet actuator includes at least one substrate with droplet operations electrodes arranged for conducting droplet operations on a surface of the substrate. For example, the droplet actuator may be open at the top, or a single substrate may be folded on itself to provide a droplet operations gap. The droplet actuator may thus include one or more substrates arranged to form a droplet operations gap and electrodes associated with one or both substrates arranged for performing droplet operations in the gap. The droplet actuator of the invention also includes one or more electroporation electrodes. In some embodiments, the electroporation electrode of the invention may be relatively flat. Lateral dimensions of the electroporation electrode may cover some portion or all of the footprint of the droplet in which electroporation is to take place. The electroporation invention may be made using a conductive material, such as a metal, such as copper or gold.

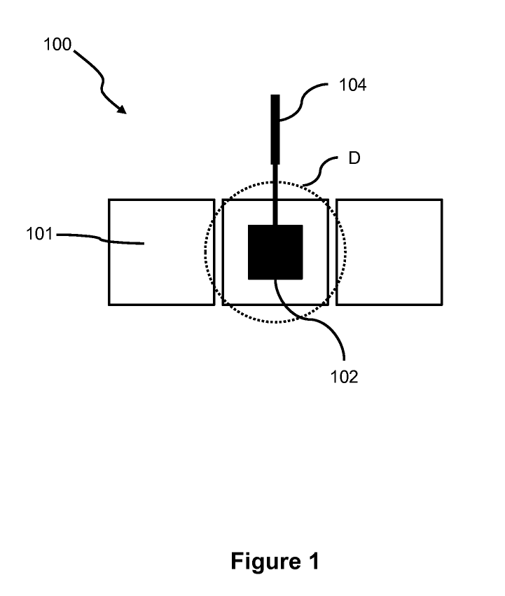

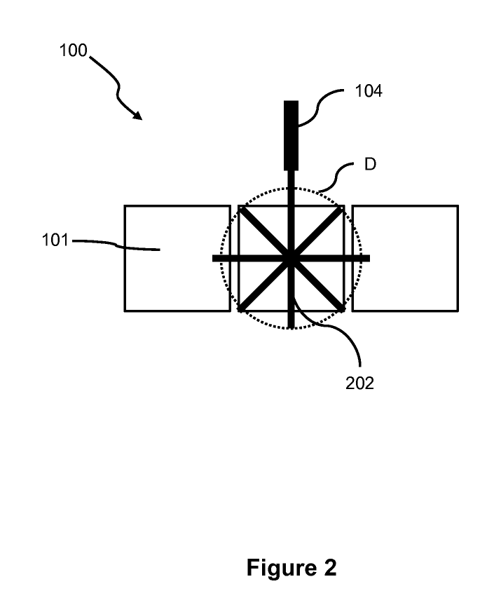

FIG. 1 illustrates a portion of a droplet operations electrode layout of a droplet actuator showing droplet operations electrodes 101 and an electroporation electrode 102 situated atop the droplet operations electrode. Conductive path 104 provides a means for coupling electroporation electrode 102 to a power source, such as an electroporation pulse generator. Droplet D is situated atop a droplet operation electrode 101 and the electroporation electrode 102. Electroporation electrode 102 may be made of any sufficiently conductive material, such as a metal, a conductive polymer, conductive ink, etc.

FIG. 2 illustrates a portion of a droplet operations electrode layout of a droplet actuator as shown in FIG. 1, except that electroporation electrode 202 has radially oriented arms that overlap an electrowetting electrode 101 and portions of adjacent electrowetting electrodes 101.

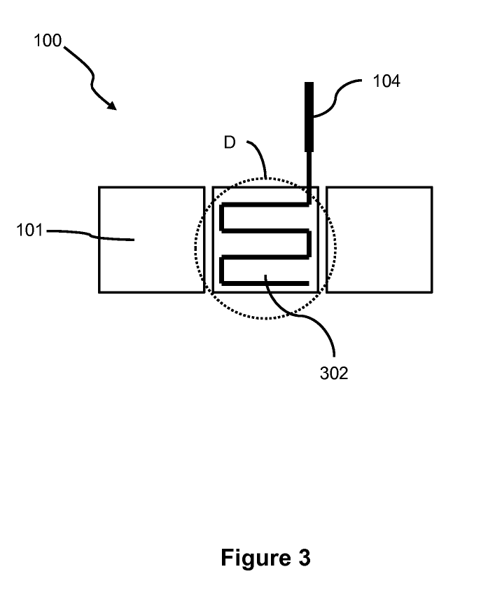

FIG. 3 illustrates a portion of a droplet operations electrode layout of a droplet actuator as shown in FIG. 1, except that electroporation electrode 302 has a serpentine shape that overlaps an electrowetting electrode 101.

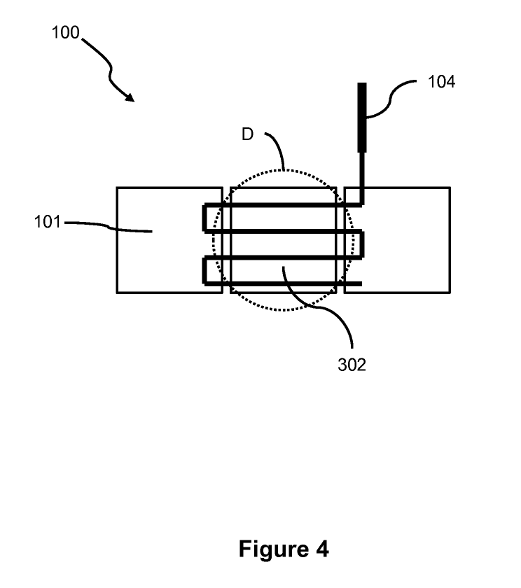

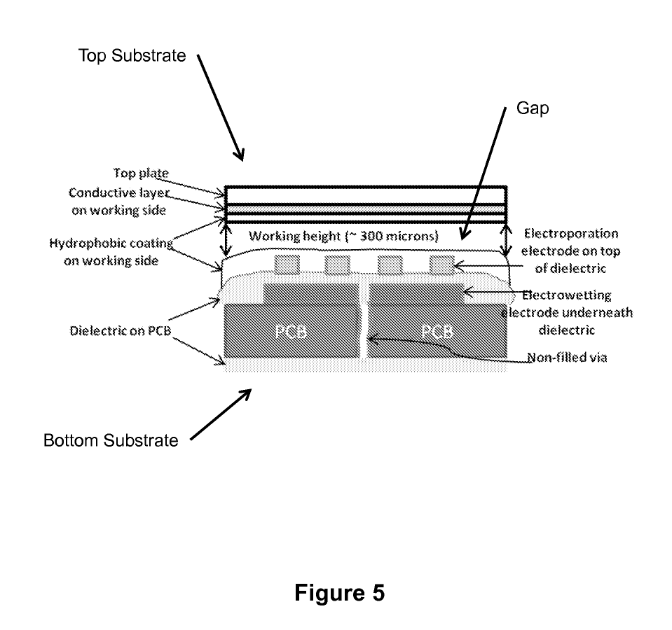

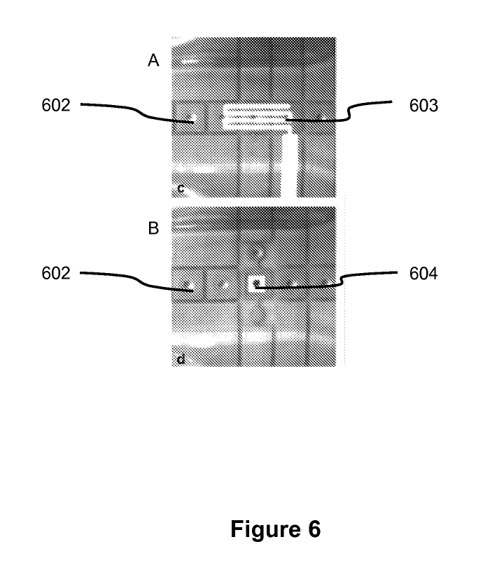

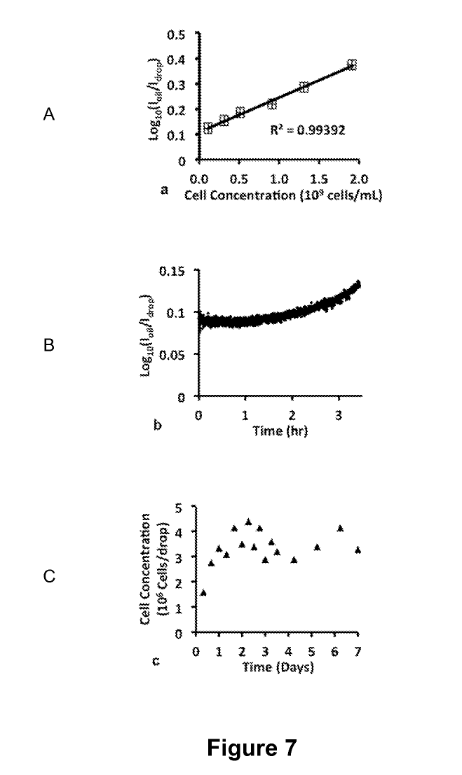

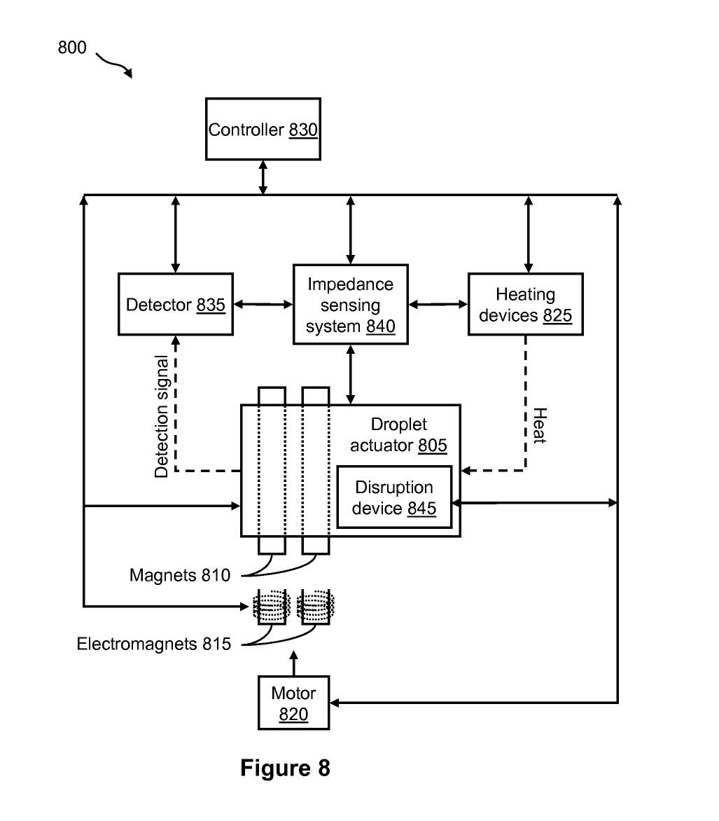

FIG. 4 illustrates a portion of a droplet operations electrode layout of a droplet actuator as shown in FIG. 1, except that electroporation electrode 402 has a serpentine shape that overlaps an electrowetting electrode 101 and portions of adjacent electrowetting electrodes 101.

Typically, the electroporation electrodes are arranged atop one or more of the droplet operations electrodes. In order to facilitate electrowetting in the presence of the electroporation electrode, it is preferable for the electroporation electrode to be formed in a manner which does not completely block the electrowetting effect produced by the underlying electrowetting electrode.

Electroporation electrodes 102, 202, 302, and 402 are examples. A skilled artisan can readily envision many more embodiments in view of this disclosure. By providing the electroporation electrode in an arrangement which does not completely block the electrowetting effect, the underlying electrowetting electrode remains functional for conducting droplet operations to manipulate droplets atop the electroporation electrode. For example, the invention provides designs and methods which permit a droplet to be transported along a path of electrowetting electrodes onto an electroporation electrode.

FIG. 5 illustrates one embodiment of the invention. The figure shows a cross-section of a portion of a droplet actuator of the invention having electrophoresis capabilities. The droplet actuator has a top substrate and a bottom substrate. The bottom substrate may be formed of a PCB, plastic, glass, semiconductor materials, or other dielectric materials. An electrowetting electrode is shown atop the bottom substrate. A via extends through the substrate or into the substrate, and provides a means for electrically coupling the electrode to a power source for activation of the electrode. A dielectric layer is applied atop the electrodes and atop the bottom substrate in areas between the electrodes. The electroporation electrode is deposited atop the dielectric material. A hydrophobic coating overlies the electroporation electrode and the dielectric material surrounding the footprint of the electroporation electrode. The top substrate is separated from the bottom substrate by a gap. The electrodes on the bottom substrate face the gap. The top substrate may, for example, be formed from a PCB, plastic, glass, semiconductor materials, or other dielectric materials. On the gap-facing side of the top substrate, a conductive layer is applied. A hydrophobic coating overlies the conductive layer. The gap may be referred to as a droplet operations gap. Droplet operations may be conducted in the droplet operations gap. The electrode illustrated here may be part of a path or an array of electrodes.

### 8.2 DROPLET OPERATIONS AT ELECTROPORATION ELECTRODES

The invention provides a method of producing electroporation in a droplet comprising situating a droplet atop an electroporation electrode which is covered with a hydrophobic coating, and applying an electroporation pulse to the electroporation electrode. Situating the droplet atop the electroporation electrode may be accomplished using droplet operations, such as electrode-mediated droplet operations, such as electrowetting mediated droplet operations.

The invention provides a method of producing electroporation in a droplet comprising situating a droplet in oil atop an electroporation electrode which is covered with a hydrophobic coating, and applying an electroporation pulse to the electroporation electrode. In this and other embodiments, the droplet may be surrounded by oil. In this and other embodiments, the droplet may be substantially surrounded by the oil. In this and other embodiments, the droplet may be floating in the oil. A wide variety of oils are known in the art for use in electrowetting. Situating the droplet atop electroporation electrodes may be accomplished using droplet operations, such as electrode-mediated droplet operations, such as electrowetting mediated droplet operations.

The invention provides a method including:

1. Transporting by electrowetting a droplet including cells along a path of electrowetting electrodes onto an electroporation electrode;
2. Delivering a pulse to the electroporation electrode thereby causing electroporation of cells in the droplet; and
3. Transporting by electrowetting away from the electroporation electrode.

The electroporation may result in transformation of one or more cells in the droplet.

Activation of an electroporation electrode in the presence of a droplet may cause pinning Pinning occurs when the droplet cannot be transported away from the electroporation electrode using settings that were used to transport the droplet onto the electroporation electrode. The invention provides droplet actuator designs and techniques which eliminate or minimize or reduce the effect of pinning. For example, the invention provides designs and methods which permit a droplet to be transported away from the electroporation electrode.

In one embodiment, a pinned droplet is transported away from the electroporation electrode by adjusting the electrowetting frequency. For example, the electrode is typically activated at 300 V 30 Hz for electrowetting. However, reducing the frequency, e.g., to 2 Hz, can reverse pinning, permitting the droplet to be transported away from the electrode.

While not wishing to be bound by a particular theory, the inventors hypothesize that this reversal in pinning may result from oscillation caused in the droplet at 2 Hz. The invention thus includes a method of conducting a droplet operation using a pinned droplet by adjusting the electrode activation to a frequency which causes sufficient oscillation to reverse the pinning effect. Similarly, the invention includes a method of transporting a pinned droplet by adjusting the electrode activation to a frequency which causes sufficient oscillation to reverse the pinning effect.

Transport of a pinned droplet can also be improved by reducing the transport rate, i.e. the rate at which the droplet is transported from one electrode to the next. For example, the typical transport rate is 1 second. The inventors have determined that a 30 second transport rate can be effective to transport a droplet away from a pinned position. The invention includes a method of transporting a pinned droplet by adjusting the transport rate to a rate which results in transport of the droplet. Similarly, the invention includes a method of transporting a pinned droplet by reducing the transport rate to a rate which results in transport of the droplet. Similarly, the invention includes a method of transporting a pinned droplet by reducing the transport rate to greater than about 1 second, or greater than about 5 seconds, or greater than about 10 seconds, or greater than about 20 seconds, or greater than about 30 seconds.

Examples of techniques for mitigating transport failures to and/or from electroporation electrodes:

1. Use a droplet having a size which is approximately two times the footprint of the electrowetting electrode (2x droplet (e.g., ˜700 nL) instead of a 1× droplet (˜350 nL));
2. In order to overcome shielding of the electrowetting electrodes by the electroporation electrode on top of the dielectric layer, use a 5 second transport rate instead of 1 second transport rate at 300 V 30 Hz electrowetting voltage and transport 2× droplet as a combination of 1× and 2× to park the 2× droplet efficiently on top of electroporation electrode (e.g., when transporting the droplet to the electroporation electrode, the transport rate is slowed to at least 5 seconds per electrode and the droplet is transported in a “slug” type movement by alternating between a 1× droplet and a 2× droplet from one electrowetting electrode to the next);
3. Use electrowetting to retain the droplet in place so that droplet does not float away during electroporation pulsing;
4. Use lower electrowetting frequencies (for example 2 Hz instead of 30 Hz) post pulse to mitigate pinning and enable transport of pulsed droplet; and/or
5. Use at least 30 second transport rate/electrode and use a combination of 1× and 2× transport modes to mitigate pinning and enable transport of pulsed droplet (e.g., transporting the droplet in a “slug” type movement by alternating between a 1× droplet and a 2× droplet from one electrowetting electrode to the next, combined with the transport rate of at least 30 seconds per electrode and oscillation of the droplet at low frequency, provides enough movement of the droplet to overcome droplet pinning).

### 8.3 DOWNSTREAM PROCESSING

Following electroporation, the droplet can be transported away from the electroporation electrode and may be subjected to further downstream processing.

In one embodiment, following electroporation using a droplet actuator of the invention, the droplet is promptly merged with a recovery media. The resulting combined droplet may be incubated and then subjected to further processing on the droplet actuator or removed from the droplet actuator.

In another embodiment, the droplet may be split into two or more daughter droplets, each calculated to contain a single cell, and the daughter droplets may be combined with a culture medium and incubated to grow the cells.

In yet another embodiment, the droplet may be combined with a larger droplet to dilute its contents, and the resulting droplets may be split into daughter droplets, each calculated to contain a single cell. Again, the daughter droplets may be combined with culture medium and incubated to grow the cells.

Incubated droplets may be sampled by dispensing daughter droplets from the incubated droplet. The sample droplets may be subjected to assays or other testing to identify and/or quantify their contents or certain aspects of their contents. For example, the daughter droplets may be tested to determine whether the electroporation achieved the desired transformation of the cells.

Incubated droplets may be periodically refreshed with fresh culture media, e.g., by transporting a droplet of culture media into contact with the incubated droplet.

In another embodiment, the electroporation electrode is situated adjacent to a recovery port, and a droplet of recovery media is present at the recovery port. In this embodiment, immediately following electroporation, the droplet subject to electroporation is immediately transported via electrowetting off of the electroporation electrodes and into proximity with the recovery droplet. The resulting combined daughter droplet can then be incubated if desired and then removed from the droplet actuator via the port. The recovery port may, for example, be an opening in the top substrate or the bottom substrate or a sidewall of the droplet actuator.

In one embodiment, the invention provides for multiplex automated genome engineering (MAGE), a process that allows for a large-scale programming and directed evolution of cell lines through the repeated introduction of synthetic DNA using the electroporation techniques of the invention.

### 8.4 EXAMPLE

The inventors have demonstrated bulk cell transformation by an electroporation device integrated with a digital microfluidics system, which achieved up to 2% transformation efficiency while maintaining fluid transport capability. Towards the goal of enabling efficient MAGE cycling with real time feedback control, monitoring of cell recovery and growth was implemented via reflectance measurements with a limit of detection of about 108 cells/ml. Furthermore, simulated MAGE cycles were performed and showed that cells remained viable for at least 16 cycles on-chip.

Electroporation: EcNR2 cells were grown off-cartridge to mid-log growth phase, then separated from the growth medium and re-suspended in de-ionized (DI) water with 10 ng/μL GalK recovery oligonucleotides. Droplets of oligonucleotides and cells were dispensed and actuated to the electroporation electrodes. FIG. 6 is two photograph A and B, each showing a portion of the top side of the bottom substrate 601 of a droplet actuator of the invention, showing the droplet operations electrodes 602 and two electroporation electrodes, a serpentine electroporation electrode 603 and a square electroporation electrode 604.

An exponentially decaying pulse (I=6 ms, 1 kV peak-voltage) was applied to the droplets with a Bio-Rad Micropulser. Droplets were then actuated to recovery reservoirs containing galactose-rich LB growth medium for 3 hours. Transformation efficiency was evaluated as the ratio of transformed cells to survived cells. A maximum of 2% was achieved. During recovery, turbidity measurements were made using an Ocean Optics spectrometer, a reflectance probe and a white light source.

Cell concentration was calibrated against the logarithm of the ratio of reflected light measured through a droplet to that measured through silicone oil using cell suspensions of known concentrations. FIG. 7, Panel A shows a turbidity calibration curve relating the log ratio of oil to droplet light intensity versus cell concentration at 600 nm. FIG. 7, Panel B shows turbidity measurement during post-electroporation recovery. A lower limit of detection of ˜108 cells/ml was estimated.

MAGE Cycle Simulation: Cell morbidity associated with electroporation was simulated by repeated 128-fold dilutions on-chip. During the simulation, cells were grown for 8 hours in an on-cartridge reservoir. Aliquots of cells were taken and concentration was measured off-chip by a plate reader during each cycle. 16 cycles were achieved over a period of 7 days. FIG. 7, Panel C shows cell concentration at the end of the growth period for 16 simulated MAGE cycles over a period of 1 week.

This work demonstrates, among other things, bulk electroporation in a digital microfluidics platform, cell viability in the system for at least 16 simulated MAGE cycles, and optical measurement capabilities designed to monitor on-chip cell growth.

### 8.5 SYSTEMS

FIG. 8 illustrates a functional block diagram of an example of a microfluidics system 800 that includes a droplet actuator 805. Digital microfluidic technology conducts droplet operations on discrete droplets in a droplet actuator, such as droplet actuator 805, by electrical control of their surface tension (electrowetting). The droplets may be sandwiched between two substrates of droplet actuator 805, a bottom substrate and a top substrate separated by a droplet operations gap. The bottom substrate may include an arrangement of electrically addressable electrodes. The top substrate may include a reference electrode plane made, for example, from conductive ink or indium tin oxide (ITO). The bottom substrate may include an electroporation electrode. The bottom substrate and the top substrate may be coated with a hydrophobic material. Droplet operations are conducted in the droplet operations gap. The space around the droplets (i.e., the gap between bottom and top substrates) may be filled with an immiscible inert fluid, such as silicone oil, to prevent evaporation of the droplets and to facilitate their transport within the device. Other droplet operations may be effected by varying the patterns of voltage activation; examples include merging, splitting, mixing, and dispensing of droplets.

Droplet actuator 805 may be designed to fit onto an instrument deck (not shown) of microfluidics system 800. The instrument deck may hold droplet actuator 805 and house other droplet actuator features, such as, but not limited to, one or more magnets and one or more heating devices, as well as one or more electroporation circuits to deliver an electroporation pulse to the electroporation electrodes. Alternatively, the electroporation pulse generator may be external to the instrument.

The instrument deck may house one or more magnets 810, which may be permanent magnets. Optionally, the instrument deck may house one or more electromagnets 815. Magnets 810 and/or electromagnets 815 are positioned in relation to droplet actuator 805 for immobilization of magnetically responsive beads. Optionally, the positions of magnets 810 and/or electromagnets 815 may be controlled by a motor 820. Additionally, the instrument deck may house one or more heating devices 825 for controlling the temperature within, for example, certain reaction and/or washing zones of droplet actuator 805. In one example, heating devices 825 may be heater bars that are positioned in relation to droplet actuator 805 for providing thermal control thereof.

A controller 830 of microfluidics system 800 is electrically coupled to various hardware components of the invention, such as droplet actuator 805, electromagnets 815, motor 820, and heating devices 825, as well as to a detector 835, an impedance sensing system 840, and any other input and/or output devices (not shown). Controller 830 controls the overall operation of microfluidics system 800. Controller 830 may, for example, be a general purpose computer, special purpose computer, personal computer, or other programmable data processing apparatus. Controller 830 serves to provide processing capabilities, such as storing, interpreting, and/or executing software instructions, as well as controlling the overall operation of the system. Controller 830 may be configured and programmed to control data and/or power aspects of these devices. For example, in one aspect, with respect to droplet actuator 805, controller 830 controls droplet manipulation by activating/deactivating electrodes.

In one example, detector 835 may be an imaging system that is positioned in relation to droplet actuator 805. In one example, the imaging system may include one or more light-emitting diodes (LEDs) (i.e., an illumination source) and a digital image capture device, such as a charge-coupled device (CCD) camera. In another example, the detector may be an electrochemical sensor integrated into the top or bottom substrate, such that a droplet in the droplet operations gap can be exposed to the sensor.

Impedance sensing system 840 may be any circuitry for detecting impedance at a specific electrode of droplet actuator 805. In one example, impedance sensing system 840 may be an impedance spectrometer. Impedance sensing system 840 may be used to monitor the capacitive loading of any electrode, such as any droplet operations electrode, with or without a droplet thereon. For examples of suitable capacitance detection techniques, see Sturmer et al., U.S. Patent Application Publication No. US20100194408, entitled “Capacitance Detection in a Droplet Actuator,” published on Aug. 5, 2010; and Kale et al., U.S. Patent Application Publication No. US20030080143, entitled “System and Method for Dispensing Liquids,” published on May 1, 2003; the entire disclosures of which are incorporated herein by reference.

Droplet actuator 805 may include disruption device 845. Disruption device 845 may include any device that promotes disruption (lysis) of materials, such as tissues, cells and spores in a droplet actuator. Disruption device 845 may, for example, be a sonication mechanism, a heating mechanism, a mechanical shearing mechanism, a bead beating mechanism, physical features incorporated into the droplet actuator 805, an electric field generating mechanism, a thermal cycling mechanism, and any combinations thereof. Disruption device 845 may be controlled by controller 830. Additionally or alternatively the cell disruption device may be an electroporation electrode of the invention.

It will be appreciated that various aspects of the invention may be embodied as a method, system, computer readable medium, and/or computer program product. Aspects of the invention may take the form of hardware embodiments, software embodiments (including firmware, resident software, micro-code, etc.), or embodiments combining software and hardware aspects that may all generally be referred to herein as a “circuit,” “module,” or “system.” Furthermore, the methods of the invention may take the form of a computer program product on a computer-usable storage medium having computer-usable program code embodied in the medium.

Any suitable computer useable medium may be utilized for software aspects of the invention. The computer-usable or computer-readable medium may be, for example but not limited to, an electronic, magnetic, optical, electromagnetic, infrared, or semiconductor system, apparatus, device, or propagation medium. The computer readable medium may include transitory and/or non-transitory embodiments. More specific examples (a non-exhaustive list) of the computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Program code for carrying out operations of the invention may be written in an object oriented programming language such as Java, Smalltalk, C++ or the like. However, the program code for carrying out operations of the invention may also be written in conventional procedural programming languages, such as the “C” programming language or similar programming languages. The program code may be executed by a processor, application specific integrated circuit (ASIC), or other component that executes the program code. The program code may be simply referred to as a software application that is stored in memory (such as the computer readable medium discussed above). The program code may cause the processor (or any processor-controlled device) to produce a graphical user interface (“GUI”). The graphical user interface may be visually produced on a display device, yet the graphical user interface may also have audible features. The program code, however, may operate in any processor-controlled device, such as a computer, server, personal digital assistant, phone, television, or any processor-controlled device utilizing the processor and/or a digital signal processor.

The program code may locally and/or remotely execute. The program code, for example, may be entirely or partially stored in local memory of the processor-controlled device. The program code, however, may also be at least partially remotely stored, accessed, and downloaded to the processor-controlled device. A user's computer, for example, may entirely execute the program code or only partly execute the program code. The program code may be a stand-alone software package that is at least partly on the user's computer and/or partly executed on a remote computer or entirely on a remote computer or server. In the latter scenario, the remote computer may be connected to the user's computer through a communications network.

The invention may be applied regardless of networking environment. The communications network may be a cable network operating in the radio-frequency domain and/or the Internet Protocol (IP) domain. The communications network, however, may also include a distributed computing network, such as the Internet (sometimes alternatively known as the “World Wide Web”), an intranet, a local-area network (LAN), and/or a wide-area network (WAN). The communications network may include coaxial cables, copper wires, fiber optic lines, and/or hybrid-coaxial lines. The communications network may even include wireless portions utilizing any portion of the electromagnetic spectrum and any signaling standard (such as the IEEE 802 family of standards, GSM/CDMA/TDMA or any cellular standard, and/or the ISM band). The communications network may even include powerline portions, in which signals are communicated via electrical wiring. The invention may be applied to any wireless/wireline communications network, regardless of physical componentry, physical configuration, or communications standard(s).

Certain aspects of invention are described with reference to various methods and method steps. It will be understood that each method step can be implemented by the program code and/or by machine instructions. The program code and/or the machine instructions may create means for implementing the functions/acts specified in the methods.

The program code may also be stored in a computer-readable memory that can direct the processor, computer, or other programmable data processing apparatus to function in a particular manner, such that the program code stored in the computer-readable memory produce or transform an article of manufacture including instruction means which implement various aspects of the method steps.

The program code may also be loaded onto a computer or other programmable data processing apparatus to cause a series of operational steps to be performed to produce a processor/computer implemented process such that the program code provides steps for implementing various functions/acts specified in the methods of the invention.

### 9 CONCLUDING REMARKS

The foregoing detailed description of embodiments refers to the accompanying drawings, which illustrate specific embodiments of the invention. Other embodiments having different structures and operations do not depart from the scope of the present invention. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

1. A droplet actuator comprising:
a. at least one substrate, wherein droplet operations electrodes are associated with the at least one substrate; and
b. one or more electroporation electrodes.

2. The droplet actuator of claim 1, wherein the droplet actuator is open at the top.

3. The droplet actuator of claim 1, wherein a droplet operations gap is formed from a single substrate folded on itself.

4. The droplet actuator of claim 1, wherein a droplet operations gap is formed from a top substrate and a bottom substrate separated by the droplet operations gap.

5. The droplet actuator of claim 1, wherein the one or more electroporation electrodes are substantially flat.

6. The droplet actuator of claim 1, wherein the one or more electroporation electrodes comprise a conductive material.

7. The droplet actuator of claim 6, wherein the conductive material is a metal.

8. The droplet actuator of claim 7, wherein the metal is copper or gold.

9. The droplet actuator of claim 1, wherein the one or more electroporation electrodes are situated atop one or more droplet operations electrodes.

10. The droplet actuator of claim 1, wherein the one or more electroporation electrodes are coupled to a power source via one or more conductive paths.

11. The droplet actuator of claim 10, wherein the power source is an electroporation pulse generator.

12. The droplet actuator of claim 1, wherein the one or more electroporation electrodes comprise radially oriented arms.

13. The droplet actuator of claim 1, wherein the one or more electroporation electrodes comprise a serpentine shape.

14. The droplet actuator of claim 1, wherein the one or more electroporation electrodes overlap one or more droplet operations electrodes.

15. The droplet actuator of claim 14, wherein the one or more electroporation electrodes overlap a droplet operations electrode and portions of adjacent droplet operations electrodes.

16. The droplet actuator of claim 1, wherein the one or more electroporation electrodes are arranged to allow the droplet operations electrodes to perform electrowetting mediated droplet operations on one or more droplets.

17. A droplet actuator comprising:
a. a top substrate and a bottom substrate separated to form a droplet operations gap;
b. droplet operations electrodes atop the bottom substrate facing the droplet operations gap;
c. a via extending into the bottom substrate, whereby the droplet operations electrodes are electrically coupled to a power source;
d. a dielectric layer atop the droplet operations electrodes and atop the bottom substrate in areas between the droplet operations electrodes;
e. an electroporation electrode atop the dielectric layer, wherein the electroporation electrode comprises a footprint; and
f. a hydrophobic coating atop the dielectric material surrounding the footprint of the electroporation electrode.

19-22. (canceled)

22. A method of producing electroporation, comprising:
a. situating a droplet comprising cells atop an electroporation electrode in a droplet actuator, wherein the electroporation electrode is covered with a hydrophobic coating; and
b. delivering a pulse to the electroporation electrode, thereby causing electroporation of the cells in the droplet.

24-55. (canceled)
